(19) United States
(12) Patent Application Publication
(10) Pub. No.: US 2018/0128815 A1
(43) Pub. Date: May 10, 2018

(54) METHOD OF SCREENING AN AGENT FOR INHIBITING RECURRENCE OR METASTASIS OF BREAST CANCER

(71) Applicants: GACHON UNIVERSITY OF INDUSTRY-ACADEMIC COOPERATION FOUNDATION, Gyeonggi-do (KR); GIL MEDICAL CENTER, Incheon (KR)

(72) Inventor: Jeong Seok Nam, Seoul (KR)

(21) Appl. No.: 14/412,081

(22) PCT Filed: Oct. 2, 2014
(86) PCT No.: PCT/KR2014/009302

§ 371 Date: Dec. 30, 2014

(30) Foreign Application Priority Data
Mar. 31, 2014 (KR) 10-2014-0038031

(51) Int. Cl. G01N 33/50; C12Q 1/6886; C12Q 1/6809; A61P 35/04; G01N 33/574

(52) U.S. Cl. CPC G01N 33/5023; C12Q 1/6886; G01N 33/57415; A61P 35/04; G01N 33/5011; C12Q 1/6809

(57) ABSTRACT

The present invention relates to a method of screening preparations for inhibiting metastasis or recurrence of breast cancer using the change in the expression level of marker genes for breast cancer stem cells, wherein their expression levels are changed by the treatment with a Wnt signaling inhibitor. The method of the present invention enables to screen preparations for preventing or treating the metastasis or recurrence of breast cancer with superior accuracy and thus the method can be widely used for the effective treatment of breast cancer.

### TECHNICAL FIELD

The present invention relates, in general, to a method of screening an agent for preventing recurrence or metastasis of breast cancer and, more particularly, to a method of screening candidate compounds that can reduce the level or activity of a gene expressed in a breast cancer-specific manner using an inhibitor of recurrence or metastasis of breast cancer.

### BACKGROUND ART

Currently, the recurrence rate of breast cancer in Korean women is increasing due to high-calorie diets, a low birth rate, high maternal age at first delivery, avoidance of breast-feeding, etc. The recurrence rate of breast cancer in Korean women is highest reaching 40% in their 40s, followed by 50s, 30s, 60s, 70s, and 20s, in this order, occurring regardless of their age. However, the survival rate of women diagnosed of breast cancer has markedly increased recently due to the improvement in detection method, mass screening, and development of therapeutic treatments over the past few decades. These days, about 80% of the breast cancer patients are being diagnosed at the initial stage of cancer, during which the survival rate is highest, and as a result, about 85% of breast cancer patients allegedly survive at least 5 years after they are diagnosed of breast cancer.

Notwithstanding the progress in diagnostic technology, about 20% of the women diagnosed of breast cancer at its initial stage have a poor 10-year prognosis, and thus they experience recurrence, metastasis, or die within this period. However, the remaining 80% of the women diagnosed of breast cancer have a favorable prognosis, and thus additional active adjuvant therapy (e.g., chemotherapy) is not required. That is, although at least some of the early-stage node-negative breast cancer patients require adjuvant chemotherapy, they must be treated after being classified according to risk groups for more appropriate treatment.

In fact, most cancer patients at the early stage can survive fairly long periods without additional treatment after surgery and/or radiation therapy. Therefore, it appears that recommending all these cancer patients an active adjuvant therapy may not be appropriate considering the serious side effects of chemotherapies. Additionally, it will be very useful to divide the breast cancer patients at their early stage into a group with a favorable prognosis and an unfavorable prognosis based on the initial diagnosis. Accordingly, there is an urgent need for the development of a method for evaluating the prognosis of breast cancer patients.

Most cancer research has been focused on the methods for analyzing breast cancer prognosis and predicting therapeutic reactions and identifying the factors involved therein. Prognostic indicators not only provide information on tumor size, lymph node state, and histological grade, but also some information on prognoses, and include numerous conventional factors such as molecular markers which may react to certain therapeutic agents. For example, the method for measuring the state of receptors for steroid hormones such as estrogen (ER) and progesterone (PR) is conventionally performed for evaluating breast cancer patients.

Hormone receptor-positive tumors will certainly react to hormonal therapies, and also considering their typically less active proliferation, the prognosis of the patients with ER+/PR+ tumors are rather favorable.

Additionally, it is known that the overexpression of human epidermal growth factor receptor 2 (HER-2/neu) is associated with an unfavorable breast cancer prognosis. At present, there is a method available for predicting the reactions to trastuzumab (Herceptin®, Genentech), an anti-Her-2/neu therapeutic antibody using the expression level of Her2/neu in breast tumor. Additionally, it is known that about one third of breast cancer is associated with a mutation in p53, a tumor suppressor gene, and the mutation relates to the increased aggression of diseases and unfavorable prognoses. Additionally, the overexpression of Ki-67, a non-histone nuclear protein and a cell proliferation marker, has been known to be associated with unfavorable prognosis of breast cancer.

Although the prognostic standard and molecular markers can predict the fate of patients and provide appropriate therapeutic methods they are not sufficient as a specific and sensitive method for evaluating the recurrence and prognosis of breast cancer, and thus there is an urgent need for the development of a novel method. The method as such, should be able to specifically distinguish the breast cancer patients with favorable request from those with unfavorable prognosis, and also high risk breast cancer patients.

Meanwhile, as a way to resolve these drawbacks, extensive research has been performed to develop a technology for evaluating the recurrence and prognosis of breast cancer using a cancer stem cell derived from breast cancer. Here, the cancer stem cell refers to an initial cell which triggers a cancer within cancer cells. It is known to exist separately from cancer cells and has the characteristics of normal stem cells. If the cancer stem cells that have caused breast cancer are present in the body of the breast cancer patient after treating breast cancer, it is possible to have a negative prognosis such as recurrence or metastasis of breast cancer due to the above cancer stem cells. Accordingly, the detection of the cancer stem cells derived from breast cancer would make it possible to evaluate the recurrence and prognosis of breast cancer. However, as of yet, the method for detecting cancer stem cells derived from breast cancer has not been developed, and thus the method utilizing the cancer stem cells derived from breast cancer is currently unavailable for use.

### SUMMARY OF THE INVENTION

The present inventors, while endeavoring to develop a method to utilize cancer stem cells derived from breast cancer, discovered a marker gene expressed specifically in cancer stem cells derived from breast cancer and confirmed that an agent for preventing metastasis or recurrence of breast cancer can be screened by measuring the change in expression level of the marker gene, thereby completing the present invention.

In order to accomplish the above objective, the present invention provides a method for screening an agent for preventing metastasis or recurrence of cancer using the change in expression level of a marker gene for a cancer stem cell derived from breast cancer.

### ADVANTAGEOUS EFFECTS

The method of the present invention can screen an agent for preventing metastasis or recurrence of breast cancer rapidly, easily and accurately and thus can be widely used in the effective treatment of breast cancer.

### DESCRIPTION OF DRAWINGS

FIG. 1 shows a Heatmap illustrating the result of analysis via Ingenuity Pathways Analysis (IPA) of a gene exhibiting a change in its expression feature cultured in a 4T1 mouse breast cancer cell line via stem cell culturing method as compared with the general cell culturing method.

FIG. 2 shows the analysis result of a gene which reduces its expression in a cancer stem cell derived from breast cancer by treating with a Wnt signaling inhibitor.

FIG. 3 shows pictures of immunofluorescence staining of cultures obtained via adhesion culturing and suspending culturing in a murine breast cancer cell line 4T1 and a human breast cancer cell line MCF7 under the condition of treatment/untreatment with a Wnt signaling inhibitor (CWP232228, JW Pharmaceutical, Korea).

### DETAILED DESCRIPTION OF THE PREFERRED EMBODIMENTS

The present inventors, while performing various studies to develop a method for evaluating the recurrence and prognosis of breast cancer by detecting a cancer stem cell derived from breast cancer, have noticed the presence of a gene expressed specifically in cancer stem cells. That is, they learned that a gene expressed specifically in cancer stem cells, in which expression level is significantly increased than the general adhesion culturing method, can be discovered when cancer cells are developed by a stem cell culturing method, and confirmed that the gene is involved in Wnt signaling pathway. In this regard, the present inventors predicted that the expression level of the marker gene essentially expressed in the cancer stem cells would significantly reduce, and confirmed the same. As a result, it was confirmed that when the Wnt signaling pathway was inhibited in cancer stem cells, the expression levels of Insulin-like growth factor 1 (IGF 1), inhibitor of DNA binding 2 (Id2), matrix metalloproteinase-2 (MMP2), matrix metalloproteinase-9 (MMP9) and Wingless-type MMTV integration site family, member 5A (Wnt5a) genes reduced significantly, and the above five kinds of genes were not expressed in breast cancer cells but only in the cancer stem cells derived from breast cancer.

Since the genes with reduced levels of expression were not expressed in the breast cancer cells but only in the cancer stem cells derived from breast cancer, it was analyzed that the above five kinds of genes can be used for screening an agent capable of preventing recurrence or metastasis of breast cancer.

In an aspect of the present invention to achieve the above-described objective, there is provided a method for screening an agent for inhibiting recurrence or metastasis of breast cancer including: treating cancer stem cells derived from breast cancer with a candidate compound expected to be capable of preventing or treating recurrence or metastasis of breast cancer by cancer stem cells derived from breast cancer; and measuring the levels of mRNAs of genes selected from the group consisting of IGF1, Id2, MMP2, MMP9, Wnt5a, and a combination thereof or proteins expressed therefrom in the treated cancer stem cells.

Specifically, the method of screening an agent for preventing or treating recurrence or metastasis of breast cancer of the present invention includes:

(a) treating cancer stem cells derived from breast cancer with a candidate compound expected to be capable of preventing or treating recurrence or metastasis of breast cancer;

(b) measuring the levels of mRNAs or a protein encoded by at least one gene selected from the group consisting of Insulin-like growth factor 1 (IGF 1), Inhibitor of DNA binding 2 (Id2), Matrix Metalloproteinase-2 (MMP2), Matrix Mmetalloproteinase-9 (MMP9) and Wingless-type MMTV integration site family, member 5A (Wnt5a) in the cells treated with the candidate compound; and

(c) selecting the candidate compound capable of reducing the level of mRNA or a protein encoded by the gene compared to that of a negative control group not treated with the candidate compound.

In particular, the levels of mRNAs or proteins expressed therefrom for each of the above genes can be measured using a preparation for measuring proteins or a composition or a kit including the above preparation. When the measured level of an experimental group is significantly lower than that of the control group, the candidate compound may be determined as an agent to be used for preventing or treating the recurrence or metastasis of breast cancer.

As used herein, the term “breast cancer cell” refers to a cell derived from the cancer tissue of a breast cancer patient, and may be used to have the same meaning as a breast cancer cell line which, being immortalized, can indefinitely proliferate via subculture. The breast cancer cell line may include HeLa cell, etc.

As used herein, the term “cancer stem cell” refers to a kind of a cancer cell, which forms a tumor with high efficiency when injected into an immunesuppressed mouse, and the intrinsic heterogeneity of primary tumor apparently appears in the tumor formed above, and has an indefinite regeneration capacity.

As used herein, the term “general cell culture method” refers to a method for culturing general cells having characteristics different from stem cells, and it may conventionally refer to a method to form a single layer by allowing it to adhere to the bottom of a culture container.

As used herein, the term “stem cell culture method” refers to a method for culturing stem cells having characteristics different from general cells, and it may conventionally refer to a method to culture by suspending without allowing it to adhere to the bottom of the container. For example, in the present invention, 4T1 murine breast cancer cell line was inoculated into a DMEM medium containing EGF, bFGF, heparin and B27, and cultured by suspending it at 37° C. with 5% CO₂ for 7 days and obtained cancer stem cell in the form of spheres.

As used herein, the term “IGF1 gene” refers to a gene encoding insulin-like growth factor 1, wherein the protein serves as a receptor for growth hormone thereby mediating the growth reaction of the body by the growth hormone. The specific nucleotide sequence and protein information of the gene is published in the NCBI (GenBank: NM_000618).

As used herein, the term “Id2 gene” refers to a gene encoding DNA-binding protein inhibitor 2, wherein the protein serves as a transcription regulator including a helix-loop-helix (HLH) domain. The specific nucleotide sequence and protein information of the gene is published in the NCBI (GenBank: NM_002166).

As used herein, the term “MMP2 gene” refers to a gene encoding matrix metalloproteinase-2, wherein the protein serves to decompose extracellular matrices in the cellular physiological reactions such as embryogenesis, angiogenesis, osteogenesis, etc. The specific nucleotide sequence and protein information of the gene is published in the NCBI (GenBank: NM_001127891).

As used herein, the term “MMP9 gene” refers to a gene encoding matrix metalloproteinase-9, wherein the protein serves to decompose extracellular matrices in the cellular physiological reactions such as embryonic development, angiogenesis, osteogenesis, etc. The specific nucleotide sequence and protein information of the gene is published in the NCBI (GenBank: NM_004994).

As used herein, the term “Wnt5a gene” refers to a gene encoding Wnt-5a, wherein the protein is involved in Wnt signaling. The specific nucleotide sequence and protein information of the gene is published in the NCBI (GenBank: NM_001256105).

As used herein, the term “a preparation for measuring mRNA level of a gene” refers to a preparation used for measuring the levels of mRNAs transcribed from target genes in order to confirm the expression of the target genes included in a sample. Preferably, it may be a probe or a primer that can specifically bind to the target genes via methods such as RT-PCR, competitive RT-PCR, real-time RT-PCR, RNase protection assay (RPA), northern blotting, and DNA chip analysis, but is not particularly limited thereto.

As used herein, the term “primer” refers to a short nucleotide sequence having a free 3′ hydroxyl group capable, which can form a base pair with a complementary template and serves as a start point for copying the template strand. DNA synthesis can initiate at a suitable temperature using a primer in the presence of a reagent for polymerization (i.e., DNA polymerase or reverse transcriptase) and four different nucleoside triphosphates.

In the present invention, the primer may refer to a primer which can be used for amplification of IGF1, Id2, MMP2, MMP9 or Wnt5a gene, and the nucleotide sequence of the primer is not limited as long as it can complementary bind to IGF1, Id2, MMP2, MMP9 or Wnt5a gene and amplified via PCR

As used herein, the term “probe” refers to a nucleic acid fragment such as RNA or DNA ranging from a few nucleotides to a few hundred nucleotides, which may be manufactured in the form of oligonucleotide probe, single stranded DNA probe, double stranded DNA probe, RNA probe, etc., or may be labeled for easy detection.

In the present invention, the probe may refer to a probe which can complementary bind to IGF1, Id2, MMP2, MMP9 or Wnt5a gene, and the nucleotide sequence of the probe is not limited as long as it can complementary bind to IGF 1, Id2, MMP2, MMP9 or Wnt5a gene.

As used herein, the term “a preparation for measuring a protein level” refers to a preparation used in a method for measuring the level of proteins included in a sample, preferably antibodies used in the methods such as western blotting, enzyme linked immunosorbent assay (ELISA), Radioimmunoassay (RIA), radioimmunodiffusion, Ouchterlony immunodiffusion, rocket immnunoelectrophoresis, tissue immunostaining, immunoprecipitation assay, complement fixation assay), fluorescence-activated cell sorting (FACS) and protein chip assay.

As used herein, the term “antibody” refers to a proteinaceous molecule which can bind specifically to an antigenic region of a protein or peptide molecule. The antibody may be manufactured by a conventional method from a protein, which is obtained from an expression vector into which a marker gene encoding the protein is cloned according to the conventional method. The type of the antibody may not be particularly limited but any immunoglobulin antibody such as a polyclonal antibody, a monoclonal antibody, or a part thereof having an antigenic binding may be included, and a specific antibody such as a humanized antibody may be also included. Additionally, the antibody includes a complete form of an antibody having two entire length light chains and two entire length heavy chains, and also a functional fragment of the antibody molecule. The functional fragment of the antibody refers to a fragment having at least an antigen-binding function, and may include Fab, F(ab′), F(ab′)₂ and Fv.

In the present invention, the antibody may refer to an antibody which can specifically bind to a protein expressed from IGF1, Id2, MMP2, MMP9 or Wnt5a gene, preferably a polyclonal antibody, a monoclonal antibody, or a part thereof; that can specifically bind to each of the proteins.

Meanwhile, in performing the method of the present invention, the kit, including a preparation for measuring the level of mRNA of each of the genes to be used or the level of a protein expressed therefrom, may further include at least one different kind of a constituent composition, a solution or a device, in addition to a primer, a probe or an antibody for measuring the level of mRNA of each of the genes or the protein expressed therefrom. In an exemplary embodiment, the kit for measuring the mRNA expression level of IGF 1,

Id2, MMP2, MMP9 or Wnt5a gene may refer to a kit including essential factors for performing RT-PCR. An RT-PCR kit may include a test tube or any other suitable container, a reaction buffer (with various pH and magnesium concentrations), deoxynucleotides (dNTPs), an enzyme such as Taq-polymerase and reverse transcriptase, DNase, RNAse inhibitor, DEPC-water, sterile water, etc., in addition to each of the primer pair specific to each of the genes. Additionally, the kit may also include a primer pair specific to the gene used in the quantitative control.

In another exemplary embodiment, the kit of the present invention may include essential factors for performing DNA chip analysis. The kit for DNA chip analysis may include a substrate, to which cDNA corresponding to a gene or its fragment is attached, a reagent for manufacturing a fluorescence-labeled probe, a preparation, an enzyme, etc. Additionally, the substrate may include quantitative control gene or cDNA corresponding to its fragment.

In a further exemplary embodiment, the kit of the present invention may refer to a kit for the analysis of a protein chip for measuring the level of the protein expressed from IGF1, Id2, MMP2, MMP9 or Wnt5a gene, although not particularly limited thereto, and may include a substrate a suitable buffer, a secondary antibody labeled with a chromogenic enzyme, or a fluorescent material, a chromogenic substrate, etc. The base, although not particularly limited thereto, may include a 96-well plate synthesized with polyvinyl resin, a 96-well plate synthesized with polystyrene, a slide glass made of glass, etc. The chromogenic enzyme, although not particularly limited thereto, may include peroxidase, alkaline phosphatase, etc. The fluorescent material, although not particularly limited thereto, may include FITC, RITC, etc. The chromogenic substrate solution, although not particularly limited thereto, may include 2,2′-azino-bis(3-ethylbenzothiazolin-6-sulfonic acid) (ABTS), o-phenylenediamine (OPD), or tetramethylbenzidine (TMB).

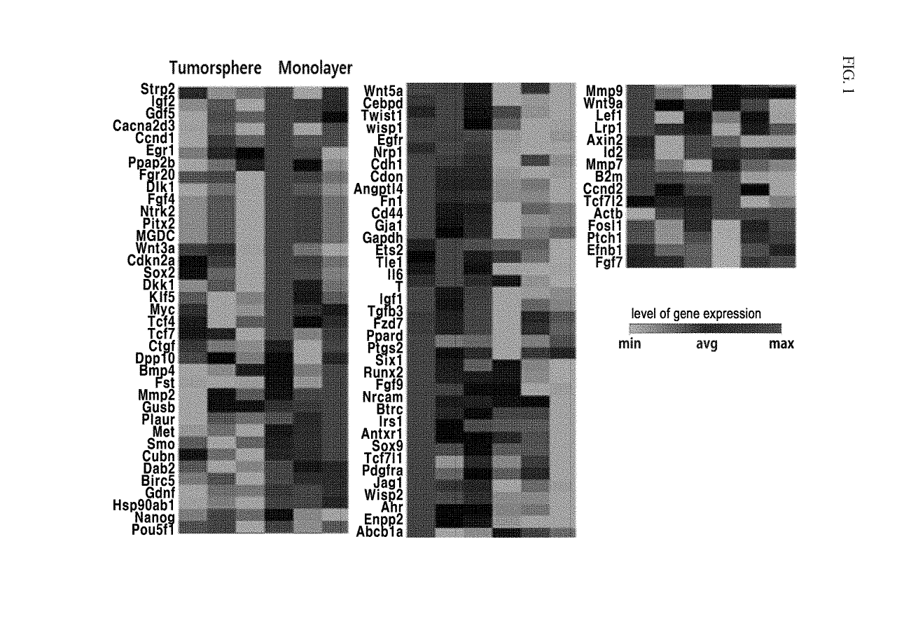

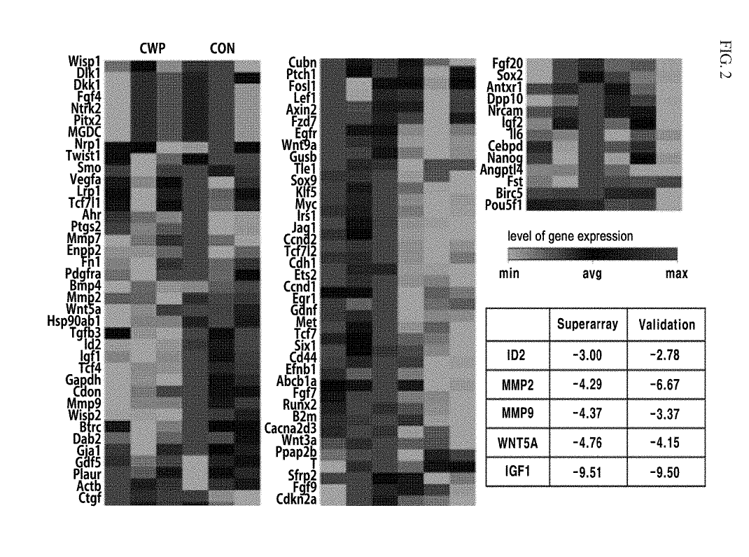

According to an embodiment of the present invention, culture products were obtained from a murine breast cancer cell line by the general culture method and the stem cell culture method, respectively, and the levels of gene expression expressed in the cultured products were compared. As a result, it was confirmed that the expression level of Wnt signaling-related gene in the cultured product by stem cell culture method was significantly increased (Example 1). Additionally, when the Wnt signaling was inhibited to select the genes whose expression level is reduced in cancer stem cell, IGF1, Id2, MMP2, MMP9 or Wnt5a gene was selected, and the selected genes were not expressed in breast cancer cells but specifically expressed only in the cancer stem cells derived from breast cancer. Additionally, when the Wnt signaling was inhibited in stem cells it was confirmed that their expression levels decreased (FIGS. 1 and 2). Accordingly, it was confirmed that the above five kinds of genes can be used as marker genes for cancer stem cells.

Conclusively, it was confirmed that the use of the preparation capable of measuring the level of mRNA of IGF1, Id2, MMP2, MMP9 or Wnt5a gene, or a protein expressed therefrom, can be used for screening the inhibitors capable of preventing the recurrence or metastasis of breast cancer by the cancer stem cells derived from breast cancer.

The present invention will be explained in greater detail through the following examples as set forth herein below, but they are disclosed for illustrative purposes only and are not to be construed as limiting the scope of the present invention.

### EXAMPLE 1

### Discovery of Signaling Genes Specific to Cancer Stem Cells Derived from Breast Cancer

### EXAMPLE 1-1

### Culturing Breast Cancer Cells

First, a 4T1 murine breast cancer cell line was added with a DMEM (Invitrogen) medium containing 10% fetal bovine serum (FBS) and 1% penicillin/streptomycin, and cultured via adhesion culture at 37° C. with % CO₂ for 3 days to obtain a culture product.

Then, the 4T1 murine breast cancer cell line was added with a DMEM medium containing 20 ng/mL of EGF, 20 ng/mL of bFGF, 4 μg/mL of heparin, and B27, and cultured via suspending culture at 37° C. with % CO₂ for 7 days to obtain a culture product in a spherical form.

### EXAMPLE 1-2

### Discovery of Signaling Genes Specific to Cancer Stem Cells Derived from Breast Cancer

Each of the culture products obtained in Example 1-1 was subjected to RNeasy Plus Mini Kit (Qiagen Inc, Valencia, Calif.) and the total RNA was extracted from each of the culture products.

The thus-extracted each of the total RNA was subjected to random hexamer and ReverAid H Minus First Strand cDNA Synthesis Kit (Thermo scientific) to synthesize their respective cDNAs. Then, RTQ-PCR (ABI 7300) was performed using the thus prepared cDNAs via Stem cell PCR array (SABioscience (www.sabiosciences.com), cat no: PAMM-405), in which primers for 84 key genes related to stem cells are included therein.

Additionally, the data obtained via RTQ-PCR was subjected to Ingenuity system program (www.ingenuity.com), and performed Ingenuity Pathways Analysis (IPA) thereby analyzing the signaling pathway, which specifically increases only in the 4T1 murine breast cancer cell line cultured via stem cell culture method (FIG. 1). As shown in FIG. 1, it was confirmed that the stem cells cultured via stem cell culture method showed a significant increase in the expression of genes involved in the Wnt signaling.

### EXAMPLE 2

### Discovery of Marker Genes for Cancer Stem Cells Derived from Breast Cancer According to Treatment with Wnt Signaling Inhibitors

As shown in the result of Example 1, based on the discovery of the significant increase in the expression of genes involved in the Wnt signaling in cancer stem cells, it was expected that the genes, whose expression levels are decreased by the inhibition of the Wnt signaling, can be used as marker genes playing important roles in cancer stem cells. Accordingly, the discovery of the genes was aimed at in this experiment.

First, 4T1 murine breast cancer cell line was cultured via the suspending culturing method in the same manner as in Example 1-1 for 8 days under the condition of with or without the treatment of the Wnt signaling inhibitor (CWP232228, JW Pharmaceutical, Korea). In particular, the Wnt signaling inhibitor was applied a total of 4 times once in two days at a concentration of 1 μM.

Then, the thus obtained culture products were subjected to RNeasy Plus Mini Kit (Qiagen Inc, Valencia, Calif.) and the total RNA was extracted from each of the culture products. The thus-extracted each of the total RNA was subjected to random hexamer and ReverAid H Minus First Strand cDNA Synthesis Kit (Thermo scientific) to synthesize their respective cDNAs. Then, RTQ-PCR (ABI 7300) was performed using the thus prepared cDNAs via Stem cell PCR array (SABioscience (www.sabiosciences.com), cat no: PAMM-405), in which primers for 84 key genes related to stem cells are included therein. The data obtained via RTQ-PCR was analyzed and, based on the 2-deltadelta ct values among the respective cells cultured, the genes with at least a 2-fold difference and a P value<0.05 were selected. As a result, IGF1, Id2, MMP2, MMP9 and Wnt5a genes were confirmed to be the genes showing a significant difference according to the treatment with the Wnt inhibitor.

Finally, in order to confirm whether the selected genes show a significant difference according to the treatment with the Wnt inhibitor was confirmed via RTQ-PCR(ABI 7300) using the primers shown below (FIG. 2).

(SEQ ID NO: 1)
1D2 F: 5′-TCT GGG GGA TGC TGG GCA CC-3′
(SEQ ID NO: 2)
1D2 R: 5′-GCT TGG GCA TCT CCC GGA GC-3′
(SEQ ID NO: 3)
MMP2 F: 5′-TTT CTA TGG CTG CCC CAA GG-3′
(SEQ ID NO: 4)
MMP2 R: 5′-GTC AAG GTC ACC TGT CTG GG-3′
(SEQ ID NO: 5)
MMP9 F: 5′-TGA GTC CGG CAG ACA ATC CT-3′
(SEQ ID NO: 6)
MMP9 R: 5′-CCA GTA CCA ACC GTC CTT GAA-3′
(SEQ ID NO: 7)
WNT5A F: 5′-ACT ATG GCT ACC GCT TCG C-3′
(SEQ ID NO: 8)
WNT5A R: 5′-GCG CTC TCA TAG GAA CCC TT-3′
(SEQ ID NO: 9)
IGF1 F: 5′-GTG GAT GAG TGT TGC TTC CG-3′
(SEQ ID NO: 10)
IGF1 R: 5′-TTT GTA GGC TTC AGT GGG GC-3′

As shown in FIG. 2, the IGF1, Id2, MMP2, MMP9 and Wnt5a genes were confirmed to show a significant difference in their expression according to the treatment with the Wnt inhibitor.

### EXAMPLE 3

### Verification of Decrease in Cellular Level Expression of Marker Genes

Whether the five kinds of genes discovered in Example 2 show a significant decrease in their expression by the treatment with the Wnt inhibitor was examined at cellular level.

In order to confirm the expression level of IGF1 gene, one of the five genes discovered above, within a cell, the 4T1 murine breast cancer cell line and MCF7 human breast cancer cell line were cultured via adhesion culture method and suspending culture method in the same manner as in Example 1 under the condition of with or without the treatment of the Wnt signaling inhibitor (CWP232228, JW Pharmaceutical, Korea) and thereby obtained the respective culture products.

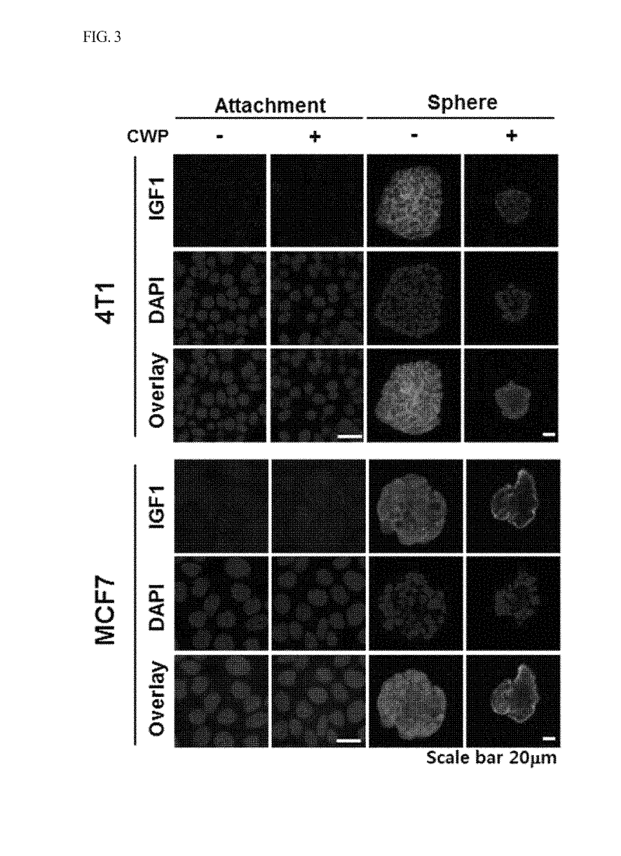

The thus obtained respective culture products were added with mouse anti-IGF1 antibodies (Milipore, cat. #05-172) to perform a primary reaction, and then added with anti-mouse IgG antibodies (Invitrogen cat.A11001) to perform a secondary reaction, thereby performing an immunofluorescence staining regarding the IGF1 gene, which was observed under an optical microscope (Zeiss LSM 510) and the expression level of IGF1 was measured (FIG. 3).

As shown in FIG. 3, IGF1 gene was not expressed in both kinds of breast cancer cell lines when they were cultured via adhesion culture method regardless of the treatment of Wnt signaling inhibitor, whereas IGF1 gene was expressed in both kinds of breast cancer cell lines when they were cultured via suspending culturing method. Therefore, it was confirmed that IGF1 gene can be used as a marker gene specific to the cancer stem cells derived from breast cancer. Additionally, the level of IGF1 gene expression was deceased in both cancer stem cells obtained from the breast cancer via suspending culture method when it was treated with the Wnt inhibitor, and the level of inhibition of IGF1 gene was significantly higher in cancer stem cells derived from murine breast cancer cell line than that of the cancer stem cells derived from human breast cancer cell line.

As described above, the five kinds of genes provided in the present invention are marker genes specifically expressed in the cancer stem cells derived from breast cancer while not expressed in the breast cancer cells. Accordingly, the genes can be widely used for screening candidate compounds capable of reducing the expression or activities of the genes with an inhibitor for metastasis and/or recurrence of breast cancer.

Although the preferred embodiments of the present invention have been described above, the present invention is not limited to the above-described specific embodiments. That is, those having ordinary knowledge in the art to which the present invention pertains can make a plurality of variations and modifications to the present invention without departing from the spirit and scope of the attached claims. All appropriate variations and modification should be construed as falling within the scope of the present invention.

1. A method of screening an agent for preventing or treating recurrence or metastasis of breast cancer, comprising:
(a) treating cancer stem cells derived from breast cancer with a candidate compound expected to be capable of preventing or treating recurrence or metastasis of breast cancer;
(b) measuring the level of mRNA or protein encoded by at least one gene selected from the group consisting of Insulin-like growth factor 1 (IGF1), Inhibitor of DNA binding 2 (Id2), Matrix Metalloproteinase-2 (MMP2), Matrix Metalloproteinase-9 (MMP9) and Wingless-type MMTV Integration site family, member 5A (Wnt5a) in the cells treated with the candidate compound; and
(c) selecting the candidate compound capable of reducing the level of mRNA or a protein encoded by the gene compared to that of a negative control group not treated with the candidate compound.

2. The method of claim 1, wherein the candidate compound is a signaling inhibitor inhibiting Wnt signaling.


SEQUENCE LISTING (10 sequences)

SEQ ID NO: 1 - DNA, 20 bp, Artificial Sequence
  primer ID2 F
tctgggggatgctgggcacc

SEQ ID NO: 2 - DNA, 20 bp, Artificial Sequence
  primer ID2 R
gcttgggcatctcccggagc

SEQ ID NO: 3 - DNA, 20 bp, Artificial Sequence
  primer MMP2 F
tttctatggctgccccaagg

SEQ ID NO: 4 - DNA, 20 bp, Artificial Sequence
  primer MMP2 R
gtcaaggtcacctgtctggg

SEQ ID NO: 5 - DNA, 20 bp, Artificial Sequence
  primer MMP9 F
tgagtccggcagacaatcct

SEQ ID NO: 6 - DNA, 21 bp, Artificial Sequence
  primer MMP9 R
ccagtaccaaccgtccttgaa

SEQ ID NO: 7 - DNA, 19 bp, Artificial Sequence
  primer WNT5A F
actatggctaccgcttcgc

SEQ ID NO: 8 - DNA, 20 bp, Artificial Sequence
  primer WNT5A R
gcgctctcataggaaccctt

SEQ ID NO: 9 - DNA, 20 bp, Artificial Sequence
  primer IGF1 F
gtggatgagtgttgcttccg

SEQ ID NO: 10 - DNA, 20 bp, Artificial Sequence
  primer IGF1 R
tttgtaggcttcagtggggc